(19) United States
(12) Patent Application Publication
(10) Pub. No.: US 2013/0224318 A1
(43) Pub. Date: Aug. 29, 2013

(54) Use of CPT-1 Modulators and Compositions Thereof

(71) Applicant: Avon Products, Inc., (US)

(72) Inventors: Cheng S. Hwang, New Milford, NY (US); Sunghan Yim, Lincoln Park, NJ (US); Uma Santhanam, Tenafly, NJ (US); John W. Lyga, Basking Ridge, NJ (US)

(73) Assignee: Avon Products, Inc., Suffern, NY (US)

(21) Appl. No.: 13/777,288

(22) Filed: Feb. 26, 2013

Related U.S. Application Data
(60) Provisional application No. 61/604,631, filed on Feb. 29, 2012.

(51) Int. Cl. A61K 8/18; A61Q 19/00

(52) U.S. Cl. CPC A61K 8/18; A61Q 19/00
USPC 424/745; 514/563; 514/545; 514/627; 514/679; 514/560; 514/456; 514/263.34; 424/758; 424/765; 424/769; 424/752; 562/444; 560/52; 564/207; 568/325; 554/224; 549/399; 544/274; 435/6.13

(57) ABSTRACT

The present invention describes methods for improving the appearance of skin, particularly, treating, ameliorating, preventing, delaying, and/or improving one or more signs of excess accumulation and/or production of subcutaneous fat, such as cellulite, and conditions related thereto, by topically applying compositions comprising Carnitine Palmitoyl Transferase-1 (CPT-1) modulators, optionally with other anti-lipid agents; and also describes compositions and methods for treating, preventing and improving the appearance of skin, particularly, treating, preventing, ameliorating, reducing and/or eliminating loss of subcutaneous fat in the skin, wherein the compositions include natural plant constituents that stimulate lipid synthesis.

### CROSS-REFERENCE TO RELATED APPLICATIONS

This application claims priority to U.S. Provisional Patent Application Ser. No. 61/604,631, filed in the United States Patent and Trademark Office on Feb. 29, 2012, the entirety of which is incorporated by reference herein for all purposes.

### FIELD OF THE INVENTION

The present invention relates generally to compositions comprising modulators of Carnitine Palmitoyl Transferase-1 (CPT-1) expression for topical application to the skin. The compositions of the present invention comprise at least one such CPT-1 modulator for providing benefits to the skin, in particular, for (1) stimulating the expression of CPT-1, an important enzyme for the oxidation of fat, and thus improving the condition and appearance of skin affected by cellulite; (2) imparting an anti-aging benefit to skin, and/or improving the aesthetic appearance of skin; and/or for (3) inhibiting the expression of CPT-1, thus stimulating lipid production (“lipogenesis”) in the skin, suitable for the treatment and prevention of the loss of subcutaneous fat, and in particular, facial fat loss, sagging skin, wrinkles, dry skin, and the like.

There is active interest in the cosmetics industry in developing products that may be applied topically to the skin to counteract adverse changes in the skin. Cosmetic products that reverse or forestall such changes are increasingly in demand. Consumers continually seek to improve the appearance of their skin and in particular to reduce visible signs of skin aging. Unwanted signs include lines and wrinkles, skin sagging or atrophy, loss of suppleness, thickness, plumpness, tautness, elasticity, resiliency, and firmness, and there remains a need for products that combat such signs of aging and, more generally, that provide anti-aging and/or anti-wrinkle effects.

Human skin is broadly divided into two layers: the surface epidermis which provides an anatomical barrier to foreign elements and maintains the body's internal environment, and the underlying dermis which provides nutritional and structural support to the epidermis. The epidermis consists of a keratinized stratified squamous epithelium comprising four types of cells: keratinocytes, melanocytes, Merkel cells, and Langerhans' cells, with the majority oepidermal cells being keratinocytes. It is comprised of several sub-layers (from the innermost outwards): Stratum germinativum/Stratum basale, Stratum spinosum, Stratum granulosum, and Stratum corneum. The keratinocytes, generated by the mitosis of keratinocyte stem cells, originate in the stratum basale and then push up through the strata. As these cells move to the surface of the skin they undergo gradual differentiation, becoming anucleated, flattened, and highly keratinized. During this process the keratinocytes become highly organized. They form desmosomes, cellular junctions, between each other and, through the excretion of keratin proteins and lipids, form an extracellular matrix which strengthens the skin. Eventually the keratinocytes die off and form the stratum corneum. The epidermis provides waterproofing and serves as a barrier to infection and other external elements. In normal and healthy skin, keratinocytes are shed and replaced continuously every 30 days. In aging skin, the stratum corneum loses its capacity to retain moisture as the rate of keratinocyte renewal is reduced, and the skin dehydrates.

Glycosaminoglycans (GAGs) are produced by the body to maintain structural integrity in tissues and to maintain fluid balance. GAGs serve as a natural moisturizer and lubricant between epidermal cells to inhibit the production of matrix metalloproteinases (MMPs)—enzymes activated by UV exposure or inflammation that contribute to the breakdown of collagen while inhibiting new collagen formation. Topical glycosaminoglycans supplements can help to provide temporary restoration of enzyme balance to slow or prevent matrix breakdown and consequent onset of wrinkle formation.

Hyaluronic acid is a type of GAG that promotes collagen synthesis, repair, and hydration and is a major component of the epidermis, where it is involved in tissue repair. When skin is exposed to excessive UVB rays, it becomes inflamed (sunburn) and the cells in the dermis stop producing as much hyaluronan, and increase the rate of its degradation. Hyaluronan degradation products then accumulate in the skin after UV exposure. HA plays an important role in the normal epidermis. In normal skin, HA is found in relative high concentrations in the basal layer of the epidermis where proliferating keratinocytes are found. Maintaining the extracellular space and providing an open, as well as hydrated, structure for the passage of nutrients are the main functions of HA in epidermis. HA content increases at the presence of retinoic acid (vitamin A). The proposed effects of retinoic acid against skin photo-damage and aging may be correlated, at least in part, with an increase of skin HA content, giving rise to increase of tissue hydration. Epidermal HA also functions as a manipulator in the process of keratinocyte proliferation, which is essential in normal epidermal function, as well as during reepithelization in tissue repair. Decrease in skin elasticity, impaired local inflammatory response, and impaired tissue repair may result from a decrease in HA levels.

The dermis is the underlying layer of the skin located between the epidermis and subcutaneous tissue. It is the thickest of the skin layers and comprises the extracellular matrix of the skin, which is maintained by fibroblast cells. Fibroblasts maintain the structural integrity of connective tissues by continuously secreting precursors of the extracellular matrix. In the aging skin, the fibroblasts which ensure a balance between the synthesis and maturation of both the collagen and elastin fibres, and their breakdown, tip this equilibrium towards the breakdown of collagen and elastin fibres.

Collagen and elastin are the major components in the dermal-epidermal junction (DEJ), i.e., a specialized structures mediating close contact between the lamina densa and the underlying connective tissue of the dermis. The dermal-epidermal junction (DEJ), interlocks forming fingerlike projections called Rete ridges. The cells of the epidermis receive their nutrients and oxygen from the blood vessels in the dermis because the epidermis does not have its own blood vessels. The Rete ridges at the DEJ increase the surface area of the epidermis that is exposed to the dermis, so that the uptake of necessary nutrients/oxygen is more efficient, and the two layers of skin can bind more strongly and resist mechanical stress. The DEJ flattens out with aging, such that the skin is more fragile and more likely to shear. This process also decreases the amount of nutrients/oxygen available to the epidermis by decreasing the surface area in contact with the dermis, thereby interfering with the skin's normal repair process and as a result, the skin shows signs of aging such as fragility, lines and wrinkles, sagging, dull, discoloration, and uneven tone, rough texture, and the like.

The main structural component of the dermis is a protein called collagen. Bundles of collagen molecules pack together throughout the dermis, accounting for three-fourths of the dry weight of skin. Procollagen is the precursor molecule of collagen, synthesized in the fibroblast, osteoblast, etc., and cleaved to form collagen extracellularly. Collagen has great tensile strength, and along with soft keratin, it is responsible for skin strength and elasticity. As aging occurs, the production of collagen is reduced, while the degradation is accelerated due to an overproduction of collagenase, i.e., protease that breaks down collagen. Collagen deficiency may lead to reduction in skin strength and elasticity, which in turn may lead to wrinkles, sagging, and fragility of the aging skin. For a more detailed background on collagen, see Lodish, et al. Molecular Cell Biology, W. H. FREEMAN, New York, NY 4.sup.th edition, 2000, the disclosures of which is incorporated herein by reference. Thus, it is anticipated that the retention of or stimulation of procollagen and/or collagen production and/or the reduction in production of collagenase would provide for a healthier and stronger skin, thereby reducing wrinkles, sagging, and fragility of the aging skin, improving the aesthetic appearance of the skin and/or imparting an anti-aging benefit to skin.

Elastin is a protein that allows the skin to stretch and recoil to its original state. It is found in the dermis layer of the skin. Elastin polymers are formed by the cross-linking of tropoelastin monomers. Although there are as many as five enzymes that can catalyze this process, it is unclear exactly how the crosslinking is regulated. Elastin is not believed to be produced past puberty, after which maintenance of the elastin polymers in tissue is regulated by competing activities of renewing (e.g., “anti-elastase”) and degrading (e.g., elastase) the elastin polymer. As one ages, an imbalance in the competing activities occurs, which results in a loss of elasticity in elastin containing tissues. This loss of elasticity in skin can appear as wrinkles in the surface of the skin. Thus, the successful restoration of youthful skin from this perspective must address a variety of key issues including: vitality of fibroblasts and keratinocytes, cell-cell adhesion in the epidermis and dermis, cell nourishment to the epidermis, cell-cell anchoring and adhesion between keratinocytes, communication between the dermis and epidermis, collagenase overproduction, collagen replacement, and mechanical properties of the skin. Any natural plant material, including an extract derived therefrom, that addresses these key issues is useful in the topical composition of the present disclosure.

Consumers continually seek to improve the appearance of their skin, and in particular seek to improve the appearance of skin affected by unwanted deposition and/or accumulation of fat, including cellulite. There is active interest in the cosmetics industry to develop products that may be applied topically to the skin to provide anti-cellulite benefits, as well as other anti-lipid benefits. Cosmetic products that enhance the appearance of skin are increasingly in demand as consumers increasingly seek to mitigate and delay signs of excess accumulation and/or production of subcutaneous fat.

Cellulite is the lumpy uneven type of subcutaneous fat that tends to accumulate on the buttocks, thighs, and limbs of many women. It is considered unsightly because it gives the tissues underlying the skin an “orange peel” or “cottage cheese” look. Compressing the skin, as when sitting or crossing the legs, produces a “mattress appearance” with bulging and pitting of the fatty layer. Nodules of fat may be felt trapped within hardened connective tissue. The histology of cellulite-affected skin indicates that cellulite results from a combination of enlarged fat tissue and weak dermal structure and connective tissue septa. Excess fat accumulation increases the volume of adipocytes, which bulge into a weakened dermis to create the characteristic irregularities in the appearance of the epidermal surface. A number of factors can cause cellulite including, e.g., hereditary, intestinal, circulatory, lymphatic, hormonal, and lifestyle factors. Dieting to decrease fat intake, exercising to increase fat metabolism and prevent the build up of cellulite, and massage and hydrotherapy to stimulate lymphatic drainage can help reduce the appearance of cellulite. Nonetheless, these means for combating cellulite or subcutaneous fat are limited, and the need remains for additional approaches.

There is a need in the art for compositions and methods for improving the appearance of skin, including the treatment, control, management, amelioration, prevention, inhibition, delay, and/or reduction of excess accumulation and/or production of subcutaneous fat, including cellulite, acne, and/or oily skin.

It is therefore an object of the invention to provide new ingredients to treat, ameliorate, prevent, inhibit, delay, and/or reduce the signs of excess accumulation and/or production of subcutaneous fat. Novel methods and compositions, as well as their mode(s) of action, are disclosed herein for treating conditions related to excess accumulation and/or production of subcutaneous fat, including cellulite, as well as skin formulations comprising same, and other personal care products for the skin.

The present invention also relates to the novel use of natural plant materials, or extracts derived therefrom, in cosmetic products for the face and body. More particularly, the present invention relates to the use of topical compositions having natural plant materials or extracts that stimulate lipid production (“lipogenesis”) in the skin. Such compositions are particularly suitable for the treatment and prevention of the loss of subcutaneous fat, and in particular, facial fat loss, sagging skin, wrinkles, dry skin, and the like. The invention further relates to methods of delivery for such compositions so as to treat, including prevent, reduce, ameliorate, and/or eliminate, signs of dermatological aging and to improve the aesthetic appearance of the skin.

The foregoing discussion is presented solely to provide a better understanding of nature of the problems confronting the art and should not be construed in any way as an admission as to prior art nor should the citation of any reference herein be construed as an admission that such reference constitutes “prior art” to the instant application.

### SUMMARY OF THE INVENTION

The protrusion of enlarged fat tissue into the dermis is one of the major factors contributing to the appearance of cellulite. One of the approaches to improve cellulite is to stimulate fat breakdown and reduce the amount of fat and/or lipids in the adipocytes, or fat cells. Carnitine Palmitoyl Transferase-1 (CPT-1) is a mitochondrial enzyme that catalyzes the conjugation of carnitine to fatty acids, which is the rate-limiting step of fatty acid oxidation (fatty acid breakdown). Without wishing to be bound by theory, it is believed that an increase in CPT-1 expression leads to a reduction in lipid accumulation in fat cells, which in turn decreases the size of adipose tissue and helps improve the appearance of skin affected by cellulite. It is further believed that a decrease in CPT-1 expression leads to an stimulation of lipid production in the skin, which in turn increases the size of subcutaneous fat tissue and helps improve the aesthetic appearance of dermatologically aged skin.

Prior to this invention, it was not known that a modulator of CPT-1 expression in human preadipocytes could reduce or stimulate lipid accumulation in fat cells, improve the aesthetic appearance of skin and/or impart anti-aging benefits Furthermore, CPT-1 modulators can be beneficial in treating other skin and/or scalp conditions characterized by excess lipids, e.g., acne or oily skin; or by insufficient subcutaneous fat, such as dermatological signs of aging.

It has surprisingly been found that compositions comprising one or more substances that modulate CPT-1 expression show superior improvement in the appearance of skin affected by unwanted fat deposition and/or accumulation, including skin affected by cellulite, when topically applied thereto. In particular, compositions that are modulators of CPT-1 in human pre-adipocytes have surprisingly been found to increase procollagen production by fibroblasts and/or reduce fat accumulation and adipocyte differentiation, offering combined mechanisms of action to combating unwanted subcutaneous fat, and cellulite in particular. Furthermore, modulators of CPT-1 can be beneficial in treating other skin conditions characterized by excess lipids, e.g., acne or oily skin.

It has further surprisingly been found that compositions comprising one or more substances that inhibit CPT-1 expression may stimulate lipid production (“lipogenesis”) in the skin. Such compositions will be particularly suitable for the treatment and prevention of the loss of subcutaneous fat, and in particular, facial fat loss, sagging skin, wrinkles, dry skin, and the like.

In one embodiment, the invention comprises a method for treating a skin condition characterized by excess lipids, comprising topically applying to skin in need thereof an effective amount of at least one Carnitine Palmitoyl Transferase-1 (CPT-1) stimulator in a cosmetically acceptable vehicle for a time sufficient to improve the appearance of said skin.

In another embodiment, the invention comprises at least one CPT-1 stimulator selected from the group consisting of bezafibrate, fenofibrate, capsaicin, curcumin, docosahexaenoic acid, (−)-epigallocatechin-3-gallate, caffeine, auraptene, R-alpha-lipoic acid, acetyl-L-carnitine, trans-10, cis-12 conjugated linoleic acid, soy isoflavones, L-carnitine, bitter melon, peroxisome proliferator-activated receptor beta/delta agonist GW501516, rexinoids, thiazolidinediones, alpha-linolenic acid, tetrahydro-4-methylene-2R-octyl-5-oxo-3S-furancarboxylic acid (C75), biguanide (buformin), genestein, inhibitors of BHLHB2 proteins, 3,5-dihydroxybenzoic acid derivatives, hydroxamic acid derivatives, and combinations thereof.

In another embodiment, the skin condition is cellulite.

In another embodiment, the skin condition is acne.

In another embodiment, the skin condition is oily skin.

In another embodiment, said at least one CPT-1 stimulator in a cosmetically acceptable vehicle further comprises at least one other anti-lipid agent.

In another embodiment, said at least one other anti-lipid agent comprises at least one agent selected from the group consisting of a phosphodiesterase inhibitor, an adenylate cyclase activator, a lipolysis stimulator, a beta-adrenergic receptor agonist, an alpha-2-adrenergic receptor antagonist, and combinations thereof.

In another embodiment, said at least one other anti-lipid agent comprises at least one agent selected from the group consisting of a xanthine analog, forskolin, a Coleus forskohlii extract, a hawthorne extract, a cola extract, isoproterenol, yohimbine, Ginkgo biloba extract, perilla oil, and combinations thereof.

In another embodiment, said cellulite is found on at least one of a thigh, buttocks, abdomen, hip, and/or upper arm region.

In another embodiment, said time sufficient to improve the appearance of said skin comprises a period of at least 2 weeks, wherein said effective amount of at least one CPT-1 stimulator in a cosmetically acceptable vehicle is applied at least once a day.

In another embodiment, said at least one CPT-1 stimulator in a cosmetically acceptable vehicle further comprises at least one collagen and/or elastin stimulator.

In another embodiment, a method for reducing the re-occurrence of cellulite in an area previously affected by cellulite is provided, comprising topically applying thereto an effective amount of at least one CPT-1 stimulator in a cosmetically acceptable vehicle, for a time sufficient to improve the appearance of said skin.

In another embodiment, a method for reducing unwanted fat deposition and/or accumulation is provided, comprising topically applying to an area of skin in need thereof an effective amount of at least one CPT-1 stimulator in a cosmetically acceptable vehicle, for a time sufficient to reduce said unwanted fat.

In another embodiment, a personal care or cosmetic composition for treating a skin condition characterized by excess lipids is provided, comprising an effective amount of at least one CPT-1 stimulator in a cosmetically acceptable vehicle.

In another embodiment, said composition further comprises at least one other anti-lipid agent selected from the group consisting of a xanthine analog, forskolin, a Coleus forskohlii extract, a hawthorne extract, a cola extract, isoproterenol, yohimbine, Ginkgo biloba extract, perilla oil, and combinations thereof.

In another embodiment, the composition further comprises at least one other anti-lipid agent selected from the group consisting of a phosphodiesterase inhibitor, an adenylate cyclase activator, a lipolysis stimulator, a beta-adrenergic receptor agonist, an alpha-2-adrenergic receptor antagonist, and combinations thereof

In another embodiment, the composition further comprises at least one other anti-lipid agent selected from the group consisting of a xanthine analog, forskolin, a forskohlii extract, a hawthorne extract, a cola extract, isoproterenol, yohimbine, Ginkgo biloba extract, perilla oil, and combinations thereof.

In another embodiment, the composition further comprises at least one collagen and/or elastin stimulator.

In another embodiment, a method for identifying CPT-1 modulators is provided, comprising:

contacting cultured adipocytes with a candidate compound;

measuring CPT-1 expression mRNA from said adipocytes; and

comparing the CPT-1 mRNA expression levels from adipocytes treated with the compound of interest to CPT-1 mRNA levels from untreated control adipoctyes,

wherein a candiate compound which decreases CPT-1 mRNA expression levels is determined to be a CPT-1 inhibitor and a candiate compound which increases CPT-1 mRNA expression levels is determined to be a CPT-1 stimulator.

In another embodiment, the cosmetic composition comprising a CPT-1 modulator is identified by the method above.

In another embodiment, the method further comprises:

(i) culturing adipocytes;

(ii) treating one portion of said adipocytes with a compound of interest, and treating another portion of said adipocytes identically but without a compound of interest as an adipocyte negative control, for a time and under conditions sufficient to provide synthesis of triglycerides in said adipocytes;

(iii) lysing said adipocytes and releasing said triglycerides;

(iv) measuring the released triglyceride levels; and

(v) comparing the released triglyceride levels from adipocytes treated with the compound of interest with the released triglyceride levels from control, wherein a suitable active ingredient is identified as a compound of interest which increases or decreases triglyceride levels from lysed adipocytes compared to the control.

In another embodiment, the composition, comprising an active ingredient for the treatment of cellulite is identified according to the above method.

In another embodiment, a method for treating a skin condition characterized by insufficient subcutaneous lipids is provided, comprising topically applying to skin in need thereof an effective amount of at least one Carnitine Palmitoyl Transferase-1 (CPT-1) inhibitor in a cosmetically acceptable vehicle for a time sufficient to improve the aesthetic appearance of said skin.

In another embodiment, the aesthetic improvement of said skin is treatment, reduction, and/or prevention of fine lines and/or wrinkles

In another embodiment, the aesthetic improvement of said skin is improvement in thickness, plumpness, and/or tautness

In another embodiment, the aesthetic improvement of said skin is increase in skin elasticity and/or resiliency.

In another embodiment, the aesthetic improvement of said skin is treatment, reduction, and/or prevention of skin sagging.

In another embodiment, the aesthetic improvement of said skin is improvement in skin firmness.

In another embodiment, a method for improving the aesthetic appearance of skin is provided comprising topically applying to skin in need thereof an effective amount of at least one compound capable of modulating Carnitine Palmitoyl Transferase-1 (CPT-1) in human pre-adipocytes in a cosmetically acceptable vehicle for a time sufficient to improve the aesthetic appearance of said skin.

In another embodiment, a method for imparting an anti-aging benefit to skin is provided comprising topically applying to skin in need thereof an effective amount of at least one compound capable of modulating Carnitine Palmitoyl Transferase-1 (CPT-1) in human pre-adipocytes in a cosmetically acceptable vehicle for a time sufficient to prevent, ameliorate, inhibit and/or reduce signs of dermatological aging of said skin.

These and other aspects of the present invention will be better understood by reference to the following detailed description.

### DETAILED DESCRIPTION

It has surprisingly been found that compositions comprising one or more substances that modulate, inhibit, and/or stimulate Carnitine Palmitoyl Transferase-1 (CPT-1) expression in human preadipocyte cells markedly improve the appearance of skin affected by unwanted fat deposition and/or accumulation and/or unwanted loss of subcutaneous fat, including skin affected by cellulite, when topically applied thereto. In particular, cosmetic compositions comprising at least one CPT-1 modulator can be used in such methods to improve the appearance of skin affected by cellulite, as well as to reduce the re-occurrence of cellulite in a previously-affected area, and/or to reduce obesity in areas affected by unwanted fat accumulation and/or deposition, as well as to improve the aesthetic appearance of skin, including treating the effects of aging, by stimulating subcutaneous lipid production in the skin.

One aspect of the present invention relates to compositions for topical application which comprise one or more CPT-1 stimulators to treat, ameliorate, inhibit, delay, reduce the incidence or risk of, and/or reduce the signs of excess accumulation and/or production of subcutaneous fat. Improving the appearance of skin affected by cellulite and/or combating signs of unwanted subcutaneous fat may include, without limitation, one or more of the following:
(a) reduction in appearance of cellulite lumpiness and/or unevenness;
(b) reduction in pitting appearance of cellulite upon squeezing;
(c) reduction in extent of area affected by cellulite;
(d) prevention or delay in recurrence of cellulite;
(e) prevention or treatment of acne;
(f) prevention or treatment of oily skin;
(g) reduction in subcutaneous fat deposition and/or accumulation;
(h) improvement in collagen deposition; and
(i) improvement in connective tissue strength.

Improvements may be measured by methods known in the art, including, for example, by consumer panel testing. Methods of improving the appearance of skin according to the invention include reducing the appearance of cellulite in skin affected thereby. Methods according to the invention also include improving the tautness or tone of skin affected by cellulite. In practice, the compositions of the invention are applied to skin in need of treatment, that is, skin which suffers from a deficiency or loss in any of the foregoing skin attributes or which would otherwise benefit from improvement in any of the foregoing skin attributes.

A “CPT-1 modulator” may bring about an effective decrease or increase in triglyceride levels by any means, e.g., by decreasing or increasing CPT-1 mRNA transcribed and/or decreasing or increasing CPT-1 protein expressed, and/or decreasing post-translational processing of CPT-1 protein in the adipocytes.

A “CPT-1 stimulator” refers to any agent that can decrease the level of triglycerides in human adipocytes via one or more pathways mediated by CPT-1. Decrease in triglyceride levels can refer to a decrease in adipocyte proliferation and/or differentiation and/or intracellular lipid and/or triglyceride production, storage, and/or accumulation in adipocytes, and/or an increase in fatty acid oxidation and/or degradation and/or lipolysis; and/or reduced expression of lipogenic genes, in vitro or in vivo, and can be measured by any means known in the art, or as described herein. For example, the CPT-1 stimulator can act to decrease triglyceride production within human adipocytes. The CPT-1 stimulator can act to decrease serum triglycerides. In some embodiments, triglyceride levels are decreased by at least about 30%, at least about 40%, at least about 50%, at least about 60%, at least about 70%, at least about 75%, or at least about 100% compared to the level of triglyceride in the absence of the CPT-1 modulator.

A “CPT-1 inhibitor” refers to any agent that can elicit an increased production of subcutaneous fat, and/or exhibit a stimulatory effect on lipid production (triglyceride production). Production of subcutaneous fat serves to smooth out the landscape, or microrelief, of the skin, thereby effecting the prevention, amelioration, reduction, and/or eradication of sagging skin, etc. caused by loss of fat.

As another example, human pre-adipocyte CPT-1 modulator's effects also can be directly measured, e.g., by measuring an increase in CPT-1 gene expression, where the CPT-1 modulator acts to increase CPT-1 gene expression within human preadipocytes and/or adipocytes and/or act to increase procollagen production within human dermal fibroblasts. In some embodiments, CPT-1 gene expression is increased by at least about 10%, at least about 20%, at least about 25%, at least about 30%, at least about 35%, at least about 40%, at least about 50%, or at least about 100%, compared to the level of CPT-1 gene expression in the absence of the CPT-1 stimulator.

As another example, CPT-1 inhibition also can be directly measured, e.g., by measuring a decrease in CPT-1 gene expression, where the CPT-1 modulator acts to decrease CPT-1 gene expression within human adipocytes. In some embodiments, CPT-1 gene expression is decreased by at least about 10%, at least about 20%, at least about 25%, at least about 30%, at least about 35%, at least about 40%, at least about 50%, or at least about 100%, compared to the level of CPT-1 gene expression in the absence of the CPT-1 inhibitor.

In one embodiment, CPT-1 is stimulated. Mechanisms of stimulation can include up-regulating an agonist of CPT-1; down-regulating an antagonist of CPT-1; increasing the stability of CPT-1 RNA and/or protein, and/or increasing dimerization of CPT-1 with ligands that effect CPT-1 activation.

In one embodiment, CPT-1 is inhibited. Mechanisms of inhibition can include down-regulating an agonist of CPT-1; up-regulating an antagonist of CPT-1; decreasing the stability of CPT-1 RNA and/or protein, and/or decreasing dimerization of CPT-1 with ligands that effect CPT-1 activation.

The CPT-1 modulator can refer to a single compound, or a number of active compounds, and/or their diastereomers or cosmetically acceptable salts, one or more of which stimulates or inhibits CPT-1.

The CPT-1 modulator is a CPT-1 stimulator in one particularly preferable embodiment.

The CPT-1 modulator is a CPT-1 inhibitor in one particularly preferable embodiment.

Examples of CPT-1 stimulators and modulators are found in the art to be relevant to fatty acid oxidation and other conditions relating to anti-lipid activity, such as obesity and/or cellulite.

Examples of CPT-1 stimulators include, but are not limited to, bezafibrate (Cabrero et al, M. Diabetes, 50(8): 1883-90, 2001), fenofibrate (Bai et al., Zhonghua Yi Xue Za Zhi 88(4):268-70, 2008), capsaicin (Lee at al., Phytother Res. 25(6):935-9, 2011), curcumin (Na et al., Nutr. Metab. Cardiovasc. Dis. 21(7):526-33, 2011), docosahexaenoic acid (Gong et al., Wei Sheng Yan Jiu. 38(6):685-7, 691, 2009), (−)-epigallocatechin-3-gallate (green tea) (Lee et al., Ann Nutr Metab., 54(2):151-7, 2009), caffeine (Yun et al., Exp Anim. 57(5):461-9, 2008), auraptene (citrus fruit), Takahashi et al., Biofactors. 33(1):25-32, 2008), R-alpha-lipoic acid and acetyl-L-carnitine (Shen et al., Diabetologia. 51(1):165-74, 2008), trans-10, cis-12 conjugated linoleic acid (Ribot et al., Br. J. Nutr. 97(6):1074-82, 2007), soy isoflavones and L-carnitine (Shin et al., Eur. J. Nutr. 45(3):159-64, 2006), bitter melon (Momordica charantia) (Chan et al., J. Nutr. 135(11):2517-23, 2005), peroxisome proliferator-activated receptor beta/delta agonist GW501516 (Dressel et al., Mol. Endocrinol. 17(12):2477-93, 2003), rexinoids and thiazolidinediones (Singh Ahuja et al., Mol. Pharmacol. 59(4):765-73, 2001), alpha-linolenic acid (Ide T., Biofactors.; 13(1-4): 9-14, 2000), tetrahydro-4-methylene-2R-octyl-5-oxo-3S-furancarboxylic acid (C75) (Aja et al., Am. J. Physiol. Regul. Integr. Comp Physiol. 294(2):R352-61, 2008), biguanide (buformin) (Sándor et al., Acta Biochim. Biophys. Acad. Sci. Hung. 12(3):217-21, 1977), genestein and L-carnitine (WO 2004/0484885), inhibitors of BHLHB2 proteins (JP 2009/167117), 3,5-dihydroxybenzoic acid derivatives (KR 2008/087273), and hydroxamic acid derivatives (KR 845511 B1). In one embodiment of the invention, a personal care or cosmetic composition for treating a skin condition characterized by excess lipids, such as obesity, cellulite, acne, or oily skin, comprises an effective amount of at least one of these CPT-1 stimulators, or a combination thereof, in a cosmetically acceptable vehicle. The CPT-1 stimulators disclosed in these references are hereby incorporated by reference. In some embodiments, the CPT-1 stimulators of the invention exclude any of the foregoing.

Examples of CPT-1 inhibitors include, but are not limited to, heteroaryl substituted piperidine derivatives (EP 1959951 B1), CPT-1 inhibitor ST1326 (WO 2009/002433), ginseng berries (WO 2008/147148), piperidine-amide derivatives (WO 2008/145596), sulfonamide derivatives (WO 2008/074692), oxirane carboxylate and other compounds (WO 2006/041922), trimetazidine and perhexiline derivatives (WO 2007/096251), sulfonamide derivatives (WO 2006/131452), bicyclic sulfonamide derivatives (U.S. 2007/0191603), indolyl derivatives (U.S. 2007/0060567), 4-trimethylammonio-butyrates (U.S. 2009/0270500), aminobutanoic acid derivatives (WO 2006/092204), malonyl-CoA, adriamycin; D,L-aminocarnitine; acylamino carnitines; decanoylcarnitine; amiodarone; 2-bromopalmitic acid; 2-bromopalmitoylcarnitine; 2-bromopalmitoyl-CoA;2-bromomyristoylthiocarnitine; emeriamine; erucic acid; erucylcarnitine; etomoxir; etomoxiryl-CoA; glyburide; hemiacetylcarnitinium chloride; hemipalmitoylcanitinium chloride; 3-hydroxy-5-5-dimethylhexanoic acid (HDH); methyl palmoxirate(methyl-2-tetradecylglycidate); 2-tetradecylglycidic acid; oxfenicine; perhexiline; 2[5(4-chloropheyl)pentyl]-oxirane-2-carboxylic acid (POCA); 2-[3-(3-trifluoromethylphenyl)-propyl]oxiran-2-carbonyl-CoA;2-[5-(4-chlorophenyl)pentyl]-oxiran-2-carbonyl-CoA; 2-(5-phenylpentyl)oxiran-2-carbonyl-CoA; 2-tetradecyloxiran-2-carbonyl-CoA; 8,N,N-diethylamino-octyl-3,4,5 -trimethoxybenzoate (TMB-8); tolbutamide; and trimetazidine. The CPT-1 inhibitors disclosed in these references are hereby incorporated by reference. In some embodiments, the CPT-1 inhibitors of the invention exclude any of the foregoing.

CPT-1 has also been implicated in modulation of long-chain fatty acyl-Co-A levels in the hypothalamus, relating to methods of reducing food intake and glucose production (WO 2004/071458).

Cosmetic compositions of the instant invention generally comprise an amount of a CPT-1 modulator effective to improve the appearance to human skin in an area to which it is topically applied.

In some embodiments, the compositions comprise an amount of a CPT-1 modulator effective to decrease adipocyte differentiation and/or intracellular triglyceride production and/or accumulation in adipocytes in the area of skin.

In some embodiments, the compositions comprise an amount of a human pre-adipocyte CPT-1 modulator effective to increase procollagen production in the treated area of skin.

In other embodiments, the compositions comprise an amount of a CPT-1 modulator effective to increase adipocyte differentiation and/or intracellular triglyceride production and/or accumulation in adipocytes in the area of skin.

In certain preferred embodiments, the cosmetic composition comprises an amount of CPT-1 modulator from about 0.001 weight % to about 50 weight % based on the total weight of the composition; preferably from about 0.01 weight % to about 25 weight % based on the total weight of the composition; more preferably from about 0.1 weight % to about 10 weight % based on the total weight of the composition, and even more preferably from about 0.1 weight % to about 1 weight %, or about 0.5 weight %, based on the total weight of the composition. The above amounts refer to an “active amount” of the CPT-1 modulator. The term “active amount” refers to the amount of CPT-1 modulator absent diluent, solvent, carrier, filler or the like. Cosmetic compositions described herein find use as anti-lipid agents, e.g., as detailed below.

Another aspect of the instant invention relates to cosmetic use of compositions comprising a CPT-1 modulator.

In one embodiment, such CPT-1 stimulator compositions act to ameliorate, inhibit, delay, reduce, and/or improve the signs of excess accumulation and/or production of subcutaneous fat, and accordingly find use as potent anti-lipid products, and in particular anti-cellulite products.

In another embodiment, such CPT-1 inhibitor compositions act to ameliorate, inhibit, delay, reduce, and/or improve the signs of subcutaneous fat loss, and accordingly find use as potent lipogenic products, and in particular aesthetic facial appearance improvement products.

An “anti-lipid” agent or product, as used herein, refers to any substance that acts to decrease triglyceride levels in adipocytes, such as by bringing about one or more of a decrease in adipocyte proliferation and/or adipocyte differentiation; a decrease in intracellular lipid and/or triglyceride production, storage, and/or accumulation, an increase in fatty acid oxidation, degradation and/or lipolysis; and/or reduced expression of lipogenic genes, in vitro or in vivo. An “anti-cellulite” agent is product, as used herein, is a substance that exerts in anti-lipid effects so as to produce a visible or palpable improvement in skin affected by cellulite.

A “lipogenic” agent or product, as used herein, refers to any substance that acts to increase triglyceride levels in adipocytes, such as by bringing about one of more of an increase in adipocyte proliferation and/or adipocyte differentiation; an increase in intracellular lipid and/or triglyceride production, storage, and/or accumulation, a decrease in fatty acid oxidation, degradation and/or lipolysis; and/or increased expression of lipogenic genes, in vitro or in vivo.

In some embodiments, a method is provided for improving the appearance of skin affected by subcutaneous fat production and/or accumulation, such as in the case of cellulite, where the method comprises topically applying to affected skin at least one CPT-1 modulator in a cosmetically acceptable vehicle. The composition will comprise an effective amount of the substance. An “amount effective” or an “effective amount” to improve appearance to the skin refers to the active amount of a CPT-1 modulator sufficient to provide a visible improvement in skin affected by unwanted subcutaneous fat when applied to the skin for a sufficient time. Such improvements include without limitation, the following:
(a) reduction in appearance of cellulite lumpiness and/or unevenness;
(b) reduction in pitting appearance of cellulite upon squeezing;
(c) reduction in extent of area affected by cellulite;
(d) prevention or delay in recurrence of cellulite;
(e) prevention or treatment of acne;
(f) prevention or treatment of oily skin;
(g) reduction in subcutaneous fat deposition and/or accumulation;
(h) improvement in collagen deposition; and/or
(i) improvement in connective tissue strength.

The compositions of the invention can be applied to skin in need of treatment, such as skin which suffers from a deficiency or loss in any of the foregoing attributes or which would otherwise benefit from the composition's anti-lipid effects, e.g., as described herein. For example, the CPT-1 modulator can be provided in a cosmetically acceptable vehicle, topically applied to a desired area of skin, and allowed to remain on the area in an amount effective to treat and/or prevent an unwanted feature or condition of the skin, and/or to improve the aesthetic appearance of the skin. Topical application facilitates targeted delivery of the active components, e.g., without the requirement of an injection or the expertise of a health practitioner. While topical compositions are a preferred embodiment according to the invention, oral formulations are also contemplated.

“Treatment” as used herein, as well as related terms such as “treat” or “treating,” refers to eradicating, reducing, ameliorating, reducing the risk or, reducing the severity of, reducing the incidence of, or reversing one or more of the unwanted features associated with the skin condition being treated, such that the consumer perceives an improvement in the appearance of the skin or other treatment benefit with respect to the condition. “Prevention” as used herein, as well as related terms such as “prevent” or “preventing,” refers to affording skin not yet affected by the condition a benefit that serves to avoid, delay, forestall, minimize, or reduce the incidence of, risk of, or recurrence of one or more unwanted features associated with the skin condition to be prevented. Such preventative benefits include, for example, delaying development and/or recurrence of the condition, or reducing the duration, severity, or intensity of one or more unwanted features associated with the condition if it eventually develops.

Cosmetic compositions taught herein can be applied topically to an area of skin affected by cellulite to improve the appearance of the skin. An improvement may involve a reduction in appearance of lumpiness and/or unevenness, characteristic of cellulite, preferably reducing what is known as the “cottage cheese” or “orange peel” look. Further, areas of cellulite tend to bulge, pit, and dimple when squeezed or compressed, as occurs when legs are crossed when seated, which can worsen the appearance of cellulite areas. In some embodiments, an improvement involves a reduction in this pitting appearance of cellulite upon squeezing, so that the look of cellulite on the legs appears reduced even when sitting with the legs crossed. An improvement may also involve reducing the visible depth and/or intensity of cellulite.

Cellulite tends to accumulate on certain body regions, e.g., on the thighs and buttocks of many women, as well as on the abdomen, hip and/or upper arm region. In some embodiments, the extent of the area affected by cellulite is reduced, such that smaller areas of the thigh, buttocks, abdomen, hip, and/or upper arm region remain visibly affected. In preferred embodiments, one of more such regions becomes free of visible signs of cellulite following treatment with a composition described herein.

In some embodiments, a method is provided for reducing the re-occurrence of cellulite in an area that was previously affected by cellulite, but showing little or no signs of cellulite. Reducing the re-occurrence refers to delaying the recurrence of any cellulite on a previously-affected area, or reducing the extent of cellulite that re-appears on the area, such that any recurrent cellulite is less noticeable than previous amounts.

Compositions for use in the method of the instant invention will comprise a CPT-1 modulator in an amount sufficient to reduce intracellular triglyceride levels in adipocites at a given area of skin when topically applied thereto. As used herein, reducing triglyceride levels and related expressions refer to a decrease in adipocyte differentiation and/or intracellular triglyceride production, storage, and/or accumulation in adipocytes, and/or an increase in fatty acid oxidation; and/or reduced expression of lipogenic genes, in vitro or in vivo, to decrease the triglyceride content in an area of skin, preferably improving skin appearance to a perceptible extent. For example, in some embodiments, the triglyceride level is decreased by at least about at least about 30%, at least about 40%, at least about 50%, at least about 60%, at least about 70%, at least about 75%, or at least about 100% compared to the level of triglycerides in the absence of the composition. Triglyceride levels in subcutaneous adipocytes can be determined by appropriate assays, e.g., in vitro assays described herein or known in the art. For example, Example 1 below provides experimental details of assays for measuring intracellular triglyceride levels in human adipocytes.

Without wishing to be bound by theory, compositions disclosed herein act by a number of mechanisms of action to effect improvement in the appearance of skin affected by unwanted subcutaneous fat. The compositions act as CPT-1 modulators. The Carnitine Palmitoyl Transferase-1 (CPT-1) enzyme, also known as carnitine acyl transferase I or CAT-1, is a mitochondrial enzyme. CPT-1 is a member of a family of enzymes called carnitine acyltransferases. Three isoforms of CPT-1 are currently known: CPT1A, CPT1B, and CPT1C. CPT-1 is associated with the outer mitochondrial membrane and mediates the transport of long-chain fatty acids across the membrane by binding them to carnitine. Its role in fatty acid metabolism makes CPT-1 important in many metabolic disorders such as type 2 diabetes and insulin resistance. Such diseases, along with many other health problems, cause free fatty acid (FFA) levels in humans to become elevated, fat to accumulate in skeletal muscle, and a decrease in the ability of muscles to oxidize fatty acids. CPT-1 has been implicated in playing a critical role in these symptoms. Its importance in fatty acid metabolism makes CPT-1 a potentially useful enzyme to focus on in the development of treatments of many other metabolic disorders as well. The CPT-1 modulator may act to break up fatty deposits, even in mature fat cells. Furthermore, this CPT-1 modulator can be beneficial in treating other skin conditions characterized by excess lipids, e.g., acne or oily skin.

Thus, without wishing to be bound by theory, compositions disclosed herein act to combat signs of cellulite via more than one mechanism of action. That is, CPT-1 modulators used in the methods provided work to decrease subcutaneous fat deposition and/or accumulation and/or decrease adipocyte differentiation. CPT-1 is an adipocyte differentiation marker, and it acts to reduce adipocyte differentiation. A stronger dermal structure reduces the likelihood of fat nodules “blebbing” between connective tissue fibers or septa, which is believed to lead to the characteristic unsightly appearance of cellulite. Further, lower levels of subcutaneous fat further reduce the likelihood of such blebbing. As cellulite is believed to result from a combination of enlarged fat tissue and weak dermal structure, combating cellulite through these multiple approaches, as described herein, can provide superior results compared with products that utilize only one approach. Accordingly, the invention provides novel mechanisms of action to improve the appearance of cellulite, and thus potent anti-cellulite compositions for use therein.

In some embodiments, the cosmetic compositions for combating signs of unwanted subcutaneous fat can further comprise additional anti-lipid agents. For example, the cosmetic composition comprising a CPT-1 modulators in an amount effective (or amounts effective) to improve the appearance of skin may further comprise at least one other anti-lipid agent, including one other anti-cellulite agent. It is contemplated that synergistic improvements may be obtained with such combinations, in some embodiments.

Exemplary anti-cellulite agents include, without limitation, phosphodiesterase inhibitors, such as xanthine analogs, caffeine, aminophylline, and theophylline; adenylate cyclase activators, such as forskolin and Coleus forskohlii extract; lipolysis stimulators, such as hawthorne extract and cola extract; beta adrenergic receptor agonists, such as isoproterenol; alpha-2-adrenergic antagonists, such as yohimbine and Ginkgo biloba extract; perilla oil (see, e.g., U.S. Pat. No. 7,410,658); carnitine and/or creatine (see, e.g., U.S. 2007/0264205 entitled “Cosmetic Composition having Carnitine Creatinate and Methods for Using”, incorporated herein by reference in its entirety). In some embodiments, additional actives may include a collagen stimulator and/or an elastin stimulator, e.g., a substance that stimulates elastin production, and/or a glycosaminoglycan enhancer. Examples of collagen, elastin and glycosaminoglycan enhancers include, e.g., fennel extract, carrot extract, and alfalfa extract. In some embodiments, the additional actives may include a collagenase inhibitor and/or elastase inhibitor. In some embodiments, the invention relates to synergistic action of one or more compositions described herein with perilla oil, e.g., to provide enhanced anti-cellulite benefits to skin.

In some embodiments, the cosmetic compositions can further comprise at least one additional procollagen, collagen and/or elastin stimulator. Such procollagen, collagen and/or elastin stimulators are effective in, for example, providing improvement in procollagen and/or collagen production and/or improvement in maintenance and remodeling of elastin.

In some embodiments, a method is provided for reducing obesity and/or increasing weight loss and/or aiding body sculpting. The method can comprise topically applying to an area affected by unwanted fat deposition an effective amount of a CPT-1 modulator, such as a CPT-1 stimulator, in a cosmetically acceptable vehicle, for a time sufficient to reduce the unwanted fat. The CPT-1 modulating activities of the composition can reduce fat accumulation and/or adipocyte differentiation, as described herein, to reduce weight, preferably in targeted areas. Such areas may be “problem areas” from which the consumer finds it difficult to lose weight by general dieting and/or exercise. Other approaches for treating unwanted fat deposition have been described and may be used with the CPT-1 modulators disclosed herein. See, e.g., WO 04/047746.

In some other embodiments, it is contemplated that compositions described herein, such as cosmetic compositions comprising a CPT-1 modulator, will exhibit one or more benefits on aesthetic appearance, selected from the following:
(a) treatment, reduction, and/or prevention of fine lines or wrinkles,
(b) reduction of skin pore size,
(c) improvement in skin thickness, plumpness, and/or tautness;
(d) improvement in skin suppleness and/or softness;
(e) improvement in skin tone, radiance, and/or clarity;
(f) improvement in procollagen and/or collagen production;
(g) improvement in maintenance and remodeling of collagen and/or elastin;
(h) improvement in skin texture and/or promotion of retexturization;
(i) improvement in skin barrier repair and/or function;
(j) improvement in appearance of skin contours;
(k) restoration of skin luster and/or brightness;
(l) replenishment of essential nutrients and/or constituents in the skin;
(m) decreased by aging and/or menopause;
(n) improvement in skin moisturization;
(o) increase in skin elasticity and/or resiliency;
(p) treatment, reduction, and/or prevention of skin sagging; and/or
(q) reduction of pigment spots.

Based on the teachings provided herein, one of skill in the art will recognize other cosmetic and/or pharmaceutical applications for the compositions described herein, and such applications are also contemplated as within the scope of the instant invention. For example, compositions described herein may also find use in personal care products, such as skin care or hair care products, where it is desirable to produce an improvement in the appearance of skin or of hair, as described herein, upon application of the product. It is contemplated, for example, that compositions described herein can find use in lotion and/or tonic formulations that decrease the appearance of cellulite and other unwanted subcutaneous fat on various surfaces of the body. It is contemplated, for example, that compositions described herein can find use in hair care formulations which improve the appearance of hair by decreasing sebum and/or oil and/or unwanted greasiness on the hair.

Personal care products for the skin according to the invention include, for example, body lotions, body tonics, and the like. Hair care products according to the invention include, for example, shampoo, conditioner, aerosol spray, pump spray, mousse, foam, solution, serum, or the hair care composition may be incorporated into a towelette.

In another embodiment, the compounds or agents (human pre-adipocyte CPT-1 modulators) are intended for oral use, including for pharmaceutical use. Pharmaceutical formulations will include pharmaceutically acceptable carriers (i.e., diluents and excipients). The pharmaceutical compositions may be included in solid dosage forms, including compressed tablets and capsules, or in liquid or powder forms. Pharmaceutical dosage forms will typically include from about 0.5 mg to about 200 mg, or from about 1 mg to about 100 mg of the CPT-1 modulator. The dosage forms may be immediate release, in which case they will typically comprise a water-soluble or dispersible carrier such as microcrystalline cellulose, mannitol, hydroxypropyl methyl cellulose, PVP or the like, or may be delayed, sustained, or modified release, in which case they may comprise water-insoluble polymers such as cellulose ethers (e.g., ethylcellulose), alone or in combination with water soluble or dispersible polymers, to regulate the rate of dissolution of the dosage form in the stomach.

In one embodiment, the composition is intended for use as a non-therapeutic treatment. In another embodiment, the composition is an article intended to be rubbed, poured, sprinkled, or sprayed on, introduced into, or otherwise applied to the human body for cleansing, beautifying, promoting attractiveness, or altering the appearance, in accordance with the US FD&C Act, sec. 201(i).

### Treatment Regimens

The invention provides methods for improving the appearance of skin by topically applying a composition comprising a preadipocyte CPT-1 modulator over an area of skin for a period of time sufficient to improve the appearance of skin, as described herein. The composition will typically be applied to the skin in accordance with a treatment regime. The treatment regimen can comprise application one, two, or three times daily for as long as is necessary to achieve desired results, such as the anti-cellulite, anti-aging, improvement in aesthetic appearance and/or restoration of subcutaneous fat benefits described herein. This treatment regimen may comprise daily application or every-other-day application for at least about one week, at least about two weeks, at least about three weeks, at least about four weeks, at least about six weeks, at least about eight weeks, at least about twelve weeks, or more. In some embodiments, the composition is applied more than once daily for the recited periods of time, for example, twice daily, preferably once in the morning and once again at night before bed. The composition preferably is massaged thoroughly onto the area to be treated, e.g., onto the thighs, buttocks, hips, abdomen, upper arms, and the like.

Chronic treatment regimens are also contemplated, e.g., with respect to prophylactic treatments aimed at forestalling one or more signs of skin aging, decrease in skin aesthetic appearance, appearance of skin cellulite or other unwanted subcutaneous fat; as well as with respect to reducing and/or preventing the recurrence of cellulite in an area previously affected thereby; as well as with respect to inducing lipogenesis in an area affected or potentially affected by the loss of subcutaneous fat. The treatment and/or prophylactic regime may also depend on concentration of the CPT-1 modulator being used, e.g., as different concentrations may produce anti-cellulite skin benefits more quickly than others. The treatment regimens according to the invention may optionally include additional exercise, diet modulation and increased water intake.

The compositions generally are topically applied to the skin for a period of time sufficient to improve the appearance of skin affected by aging, decrease in aesthetic appearance, cellulite or other unwanted subcutaneous fat. In some embodiments, the compositions are left on the skin as a “leave-on” composition, by which is meant they are applied in a formulation that is allowed to remain in the skin without being deliberately washed and/or rubbed off for a certain period of time. For example, the composition may be left on the skin for a day, overnight, or for at least about 18 hours, for at least about 12 hours, for at least about 8 hours, or for at least about 4 hours.

CPT-1 modulators may be used to formulate cosmetic compositions as known in the art. The cosmetic compositions find use in anti-cellulite and anti-lipid compositions, preferably formulated for topical application to the skin, e.g., with a cosmetically acceptable vehicle. Formulations for cosmetic products comprising CPT-1 modulators are described in more detail below.

### Formulations Comprising CPT-1 Modulators

In accordance with the invention, the CPT-1 modulators may be formulated in a variety of product forms. The CPT-1 modulators may be prepared in targeted delivery systems, e.g., creams, lotions, gels, toners, serums, transdermal patches, and the like, particularly for topical administration. For example, the invention encompasses compositions comprising a cosmetically or dermatologically acceptable formulation which is suitable for contact with living animal tissue, particularly human tissue, with virtually no or little adverse physiological effect to the user. The invention also encompasses compositions for oral delivery. Compositions embraced by this invention can be provided in any cosmetically and/or dermatologically suitable form, preferably as a lotion or cream, but also in an anhydrous or aqueous base, as well as in a sprayable liquid form. Other suitable cosmetic product forms for the compositions include, for example, an emulsion, a cream, a balm, a gloss, a lotion, a mask, a serum, a toner, an ointment, a mousse, a patch, a pomade, a solution, a spray, a wax-based stick, a towelette, a shampoo, a conditioner, and/or a foam.

In some particular embodiments, the cosmetic composition comprising a CPT-1 modulator is provided in the form of a cream for topical application to skin affected, previously-affected, or likely-to-be affected by cellulite. In some particularly preferred embodiments, the cream comprising the CPT-1 modulator is supplied along with a gel for use with the cream, for example, by following application of the cream with application of the gel to the same area of skin. The gel preferably provides tightening polymers to enhance the cellulite-reducing effects of the cream. In more preferred embodiments, the gel provides a cooling sensation to the skin when applied to the skin following application of the cream. The cream and gel may be provided in different containers, or in different compartments of the same container. In some embodiments, the cream and gel are provided in a “tube-within-a-tube” that dispenses the cream and gel together. This allows the cream and gel to be mixed upon dispensing, e.g., immediately before application to the skin.

In addition, the compositions contemplated may include one or more compatible cosmetically acceptable adjuvants commonly used and known by the skilled practitioner, such as colorants, fragrances, emollients, humectants, preservatives, vitamins, chelators, thickeners, perilla oil or perilla seed oil (WO 01/66067 to a “Method of Treating a Skin Condition,” incorporated herein by reference) and the like, as well as other botanicals such as aloe, chamomile, and the like.

Also embraced by the invention are transdermal modes of delivery, such as patches and the like, with or without suitable penetration enhancers. The methods and compositions embodied by the invention provide a means by which the CPT-1 modulator can be effectively administered in a transdermal system or device. Examples of such devices are known in the art, e.g., as disclosed in U.S. Pat. Nos. 5,146,846; 5,223,262; 4,820,724; 4,379,454; and 4,956,171, all of which are incorporated herein by reference and such descriptions are not meant to be limiting. In a preferred method, topical application is through a sustained release vehicle, carrier, or diluent, e.g., using a topically applied sustained release patch. Preferably, when a topical patch is used, the patch is applied to the desired area for extended period of time, such as, e.g., at least about 4 hours, at least about 8 hours, at least about 12 hours, at least about 16 hours, or at least about 24 hours. In some embodiments, the extended period of time is all day, e.g., from the morning to bedtime, or overnight, e.g., while the user is sleeping.

The CPT-1 modulators of the present invention are preferably contained in a cosmetically or dematologically acceptable vehicle, medium, diluent or carrier, providing a topical formulation for use in treating, ameliorating, preventing, inhibiting, delaying, and/or reducing the signs of excess accumulation and/or production of subcutaneous fat, including improving the appearance of skin affected by cellulite.

In some embodiments, the topical formulation comprises a cosmetically acceptable vehicle (medium, diluent, or carrier) that is compatible with human skin. The cosmetically acceptable vehicle may comprise an aqueous phase, an oil phase, alcohol, or aqueous/alcohol-based solutions, ointments, lotions, gels, wax-in-water emulsions, or water-in-oil, oil-in-water, or water-oil-water emulsions, e.g., having the appearance of creams, gels, microemulsions, or aerosols.

The aqueous phase is a mixture of one or more water soluble or water dispersible substances, which can be liquid, semi-solid or solid at room temperature (25° C.). The vehicle comprises, or can be in the form of, a suspension, dispersion, or solution in water or in an aqueous-alcoholic vehicle, which may contain a thickener or gellant. A person skilled in the art can select the appropriate cosmetic form, the ingredients contained therein, as well as the method for preparing it, on the basis of the knowledge that the skilled artisan possesses.

In some embodiments, the cosmetically acceptable vehicle may include an aqueous phase which may contain water, or a mixture of water and at least one hydrophilic organic solvent, in particular an alcohol, especially a linear or branched lower monoalcohol containing from 2 to 5 carbon atoms, e.g., ethanol or propanol; a polyol, e.g., propylene glycol, sorbitol, glycerol, diglycerol, panthenol, or polyethylene glycol; and mixtures thereof. This aqueous phase may represent from about 0.5 weight % to about 99.99 weight %, based upon the total weight of the composition.

In some embodiments, when the composition of the invention is in the form of an emulsion, the composition may also optionally comprise a surfactant, preferably in an amount from about 0.1 weight % to about 30 weight %, and in particular, from about 1 weight % to about 20 weight %, based upon the total weight of the composition.

In some embodiments, the composition may also comprise a thickening polymer such as an amphiphilic polyurethane, a polyacrylic homopolymer or copolymer, a polyester, and/or a hydrocarbon-based resin.

The invention also contemplates formulations that may comprise an oil phase containing oil-soluble or oil-dispersible substances, which are liquid at room temperature (25° C.) and/or oily or waxy substances that are solid at room temperature, such as waxes, semi-solids, gums, and mixtures thereof. The waxes can include hydrocarbon-based waxes, fluoro waxes and/or silicone waxes and can be of plant, mineral, animal, and/or synthetic origin. Formulations typically comprise from about 0 weight % to about 20 weight % waxes, based upon total weight. The gums used are generally high molecular weight cyclic polydimethylsiloxanes (PDMS), cellulose gums or polysaccharides, and the semi-solid materials are generally hydrocarbon-based compounds, such as, but not limited to, lanolins and derivatives thereof. This oily phase may also contain organic solvents.

Suitable oily materials that are liquid at room temperature, often referred to as oils, include hydrocarbon-based oils of animal origin such as perhydrosqualene; hydrocarbon-based plant oils such as liquid triglycerides of fatty acids of 4 to 10 carbon atoms, for instance, heptanoic or octanoic acid triglycerides, or oils such as sunflower oil, corn oil, soybean oil, grapeseed oil, castor oil, avocado oil, caprylic/capric acid triglycerides, or jojoba oil; linear or branched hydrocarbons of mineral or synthetic origin, such as liquid paraffins and derivatives thereof, or petroleum jelly; synthetic esters and ethers, in particular esters of fatty alcohols, namely, for example, isopropyl myristate, 2-ethylhexyl palmitate, 2-octyldodecyl stearate, isostearyl isostearate; hydroxylated esters such as isostearyl lactate, octyl hydroxystearate, octyldodecyl hydroxystearate, heptanoates, octanoates and decanoates of fatty alcohols; polyol esters such as propylene glycol dioctanoate, neopentyl glycol diheptanoate, diethylene glycol diisononanoate, and pentaerythritol esters; fatty alcohols containing from 12 to 26 carbon atoms such as octyldodecanol, 2-butyloctanol, 2-hexyldecanol, 2-undecylpentadecanol, oleyl alcohol; partially hydrocarbon-based fluoro oils and/or fluorosilicone oils; silicone oils such as volatile or non-volatile, linear or cyclic polydimethylsiloxanes (PDMS) that are liquid or semisolid at room temperature such as cyclomethicones and dimethicones, optionally comprising a phenyl group, for instance phenyl trimethicones, siloxanes, and mixtures thereof. These oils are usually present in an amount of about 0 weight % to about 90 weight %, preferably from about 1 weight % to about 80 weight % by weight of the oil phase.

The oil phase of the formulation may also comprise one or more cosmetically acceptable organic solvents. These solvents are present in an amount of from about 0 weight % to about 60 weight %, preferably from about 1 weight % to about 30 weight %, based on the total weight of the composition, and may be selected from the group consisting of lipophilic organic solvents, amphiphilic organic solvents, and mixtures thereof. Suitable solvents which may be used in the composition of the invention include acetic acid esters such as methyl, ethyl, butyl, amyl or 2-methoxyethyl acetate; isopropyl acetate; hydrocarbons such as toluene, xylene, p-xylene, hexane or heptane; ethers containing at least 3 carbon atoms, and mixtures thereof. In some other embodiments, the compositions can be in the form of vesicular dispersions containing ionic and/or nonionic lipids, as described above.

In yet other embodiments, the compositions are formulated into liposomes or microspheres, which can comprise other additives or substances, and/or which can be modified to more specifically target or remain at a site following administration. (See, e.g., U.S. Pat. No. 5,770,222 to Unger et al., incorporated herein by reference.)

The formulations for use in the inventive methods may further comprise any ingredient conventionally used in the cosmetics field. These ingredients include, e.g., preserving agents, aqueous phase thickeners (polysaccharide biopolymers, synthetic polymers), fatty-phase thickeners, fragrances, hydrophilic and lipophilic active agents, and mixtures thereof. The amounts of these various ingredients are those conventionally used in the cosmetics field to achieve their intended purpose, and range typically from about 0.01 weight % to about 20 weight %, based upon the total weight of the composition or formulation. The nature of these ingredients and their amounts will be selected to be compatible with the production and intended applications of the compositions, as described herein.

In some embodiments, the formulation may optionally comprise an additional particulate phase, typically present in an amount of from about 0 weight % to about 30 weight %, based upon the total weight of the composition or formulation, preferably from about 0.05 weight % to about 20 weight %, and which can comprise pigments and/or pearlescent agents and/or fillers used in cosmetic compositions.

Suitable inorganic pigments include, but are not limited to, titanium oxide, zirconium oxide and cerium oxide, as well as zinc oxide, iron oxide, chromium oxide and ferric blue. Suitable organic pigments include barium, strontium, calcium, and aluminium lakes and carbon black. Suitable pearlescent agents include mica coated with titanium oxide, with iron oxide, or with natural pigment. Fillers are normally present in an amount from about 0 weight % to about 20 weight %, based on the total weight of the composition or formulation, preferably from about 0.1 weight % to about 10 weight %. Suitable fillers include talc, silica, zinc stearate, mica, kaolin, nylon (in particular orgasol) powder, polyethylene powder, TEFLON™, starch, boron nitride, copolymer microspheres such as Expancel (Nobel Industrie), Polytrap (Dow Coming), and silicone resin microbeads (Tospearl from Toshiba).

In some particular embodiments, the compositions for topical application can be in the form of a personal care product for the skin, preferably for the thighs, buttocks, legs, hips, abdomen, limbs, upper arms, and/or other areas of the body. Non-limiting examples include creams or lotions, salves, ointments, gels, masks, artificial tanning compositions, patches, or a solid which is poured or cast as a stick or a dish, for example.

In some particular embodiments, the compositions for topical application can be in the form of a personal care product for skin subject to or potentially subject to unwanted loss of subcutaneous fat, preferably for the face, forehead, lips, neck, arms, hands, legs, knees, feet, chest, back, groin, buttocks, and the like. In a preferred embodiment, the compositions are applied to the face.

In some embodiments, the topical formulations may also include one or more antioxidants. An antioxidant functions, among other things, to scavenge free radicals from skin, protecting the skin from environmental aggressors. Examples of antioxidants that may be used in the present compositions and formulations include compounds having phenolic hydroxy functions, such as ascorbic acid and its derivatives/esters; vitamins A, C, or E; polyphenols, beta-carotene; catechins; curcumin; ferulic acid derivatives (e.g., ethyl ferulate, sodium ferulate); gallic acid derivatives (e.g., propyl gallate); lycopene; reductic acid; rosmarinic acid; tannic acid; tetrahydrocurcumin; tocopherol and its derivatives; uric acid; or any mixtures thereof. Other suitable antioxidants are those that have one or more thiol functions (-SH), in either reduced or non-reduced form, such as glutathione, lipoic acid, thioglycolic acid, and other sulfhydryl compounds. The antioxidant may be inorganic, such as bisulfites, metabisulfites, sulfites, or other inorganic salts and acids containing sulfur. Compositions of the present invention may have an antioxidant preferably from about 0.001 weight % to about 10 weight %, and more preferably from about 0.01 weight % to about 5 weight %, based on the total weight of the composition or formulation.

In some embodiments, the topical formulations may also include one or more of the following: a skin penetration enhancer, an emollient, a skin plumper, an exfoliation promoter, and an optical diffuser. Details with respect to these and other suitable cosmetic ingredients can be found in the International Cosmetic Ingredient Dictionary and Handbook, 10th Edition (2004), published by the Cosmetic, Toiletry, and Fragrance Association (CTFA), at pp. 2177-2299, which is herein incorporated by reference in its entirety.

An emollient provides the functional benefits of enhancing skin smoothness and may aid in improving the appearance of skin affected by cellulite and other unwanted subcutaneous fat. Examples of emollients include isopropyl myristate, petrolatum, isopropyl lanolate, silicones (e.g., methicone, dimethicone), oils, mineral oils, fatty acid esters, or any mixtures thereof. The emollient is preferably present from about 0.1 wt % to about 50 wt % of the total weight of the composition or formulation.

A skin plumper serves as a collagen enhancer to the skin. An example of a suitable, and preferred, skin plumper is palmitoyl oligopeptide. Other skin plumpers are collagen and/or glycosaminoglycan (GAG) enhancing agents. The skin plumper is preferably present from about 0.1 weight % to about 20 weight % of the total weight of the composition or formulation.

In some embodiments, formulations may have one or more exfoliation promoters. Suitable examples of exfoliation promoters include alpha hydroxy acids (AHA); benzoyl peroxide; beta hydroxy acids; keto acids, such as pyruvic acid, 2-oxopropanoic acid, 2-oxobutanoic acid, and 2-oxopentanoic acid; oxa acids as disclosed in U.S. Pat. Nos. 5,847,003 and 5,834,513 (the disclosures of which are incorporated herein by reference); salicylic acid; urea; or any mixtures thereof. The preferred exfoliation promoters are 3,6,9-trioxaundecanedioic acid, glycolic acid, lactic acid, or any mixtures thereof. When an embodiment of the invention includes an exfoliation promoter, the formulation may have from about 0.1 weight % to about 30 weight %, preferably from about 1 weight % to about 15 weight %, and more preferably from about 1 weight % to about 10 weight %, of the exfoliation promoter based on the total weight of the composition or formulation.

An optical diffuser is a particle that changes the surface optometrics of skin, resulting in a visual blurring and softening of, for example, lines and wrinkles, as well as lumpiness and unevenness caused by cellulite and other unwanted subcutaneous fat. Examples of optical diffusers that can be used in the present invention include, but are not limited to, boron nitride, mica, nylon, polymethylmethacrylate (PMMA), polyurethane powder, sericite, silica, silicone powder, talc, TEFLON™, titanium dioxide, zinc oxide, or any mixtures thereof. The optical diffuser is preferably present from about 0.01 weight % to about 20 weight %, based on the total weight of the composition or formulation.

In some embodiments, formulations may have one or more retinoids. Exemplary retinoids include, without limitation, retinoic acid (e.g., all-trans or 13-cis) and derivatives thereof, retinol (Vitamin A) and esters thereof, such as retinol palmitate, retinol acetate and retinol propionate, and salts thereof.

In some embodiments, formulations may have one or more sunscreen protectors. A sunscreen protects the skin from damaging ultraviolet rays. In an illustrative embodiment of the invention, the sunscreen would provide both UVA and UVB protection, by using either a single sunscreen or a combination of sunscreens. Among the sunscreens that can be employed in the present compositions are avobenzone, cinnamic acid derivatives (such as octylmethoxy cinnamate), octyl salicylate, oxybenzone, titanium dioxide, zinc oxide, or any mixtures thereof. The sunscreen may be present in an amount from about 1 weight % to about 30 weight % of the total weight of the composition. The compositions of the invention having sunscreen bring about additional improvements to the aesthetic appearance of skin, including at least one of the following: minimizing sun-burning and/or reducing redness.

In some embodiments, the formulation may also have one or more of the following cosmetic and pharmaceutical active agents, excipients, ingredients, or adjuvants: anesthetics; antibiotics, e.g., erythromycins and tetracyclines; salicylic acids; anti-allergenics; antifungals; antiseptics; anti-irritants; anti-inflammatory agents; antimicrobials; analgesics; nitric oxide synthase inhibitors; insect repellents; self-tanning agents; skin penetration enhancers; skin cooling agents; chelating agents; colorants including dyes, lakes and pigments that may be untreated or chemically surface treated to improve wetability or some other property; demulcents; emulsifiers; fragrances; humectants; lubricants; skin protectants; moisturizers' pH adjusters; preservatives; stabilizers; surfactants; thickeners; film formers; plasticizers; viscosity modifiers; vitamins; blood flow stimulators; or any mixtures thereof. The amounts of these various substances are those that are conventionally used in the cosmetic or pharmaceutical fields to achieve their intended purposes, for example, they may constitute from about 0.01 weight % to about 20 weight % of the total weight of the composition.

Emulsifiers are typically present in the compositions or formulations of the invention in an amount from about 0.01 weight % to about 30 weight %, and preferably from about 0.5 weight % to about 30 weight %, based on the total weight of the composition or formulation. In some other embodiments, the composition or formulation is free or substantially free of emulsifiers.

Non-limiting examples of suitable thickening agents include xanthan gum, hydroxypropyl cellulose, hydroxyethyl cellulose, carbomer, gum acacia, Sepigel 305 (available from Seppic Co., France), and clays such as magnesium aluminum silicate.

The topical compositions of the present invention may include, and their utility can be enhanced, by one or more humectants, such as ureas, pyrrolidone carboxylic acids, amino acids, sodium hyaluronates, certain polyols, and other compounds with hygroscopic properties.

The general activity and mildness to skin of the present compositions can also be enhanced by neutralization to a pH from about 3.5 to about 8.0, most preferably a pH from about 3.5 to about 5.5. This neutralization is preferably accomplished with one or more of ammonium hydroxide, potassium hydroxide, sodium hydroxide, arginine or other amino acids, and/or triethanolamine.

All terms used herein are intended to have their ordinary meaning unless otherwise provided. As used herein, “% by weight” or “% wt” refers to the weight percent of a component in relation to the total weight of the composition or formulation (i.e., including any carriers, vehicles, solvents, emollients, fillers, or other components added before application to the skin) unless otherwise specified.

### EXAMPLES

### Example 1

### Assay for CPT-1 Gene Expression

Human pre-adipocytes are allowed to differentiate into adipocytes in Adipocyte Differentiation Medium for 7 days. On Day 8, Adipocyte Differentiation Medium is replaced with Adipocyte Maintenance Medium containing potential CPT-1 modulating compounds for another 7 days as described above. Test compounds at the indicated weight percentages are added every other day. At the end of treatment, RNA is extracted from the adipocytes using RNA Easy mini kit (Qiagen, CA). 200 ng of total RNA is used to generate 20 microL of cDNA using High Capacity cDNA Reverse Transcript Kit (Applied Biosystem: Cat# 4368814). Reverse transcriptase, Buffer, dNTP, Random primer, and RNase Inhibitor are diluted with the RNA according to the protocol from the manufacturer. One microliter of cDNA is used in 20 microL RTq-PCR reactions. Briefly, 10 μl of TaqMan Gene Expression Master Mix (Applied Biosystem; Cat# 4369016), 8 μl of H₂O, 1 μl of cDNA, and 1 μl of either CPT-1 primer (Applied Biosystem; Hs03046298_s1) or 18S (Applied Biosystem; 4333760-1001032) as a house keeping gene are mixed in a 96 well polypropylene plate (Agilent Technologies; Cat# 410088). RTq-PCR conditions are an incubation step at 50° C. for 2 minutes and an enzyme activation step at 95° C. for 10 minutes; followed by 45 cycles of 95° C. for 30 seconds and 60° C. for 1 minute. CT value is obtained from the software of the Stratagene MX2005P.

All samples are run in triplicate and normalized to 18S, and results are expressed as a percentage of the control.

Compounds that show a significant change in CPT-1 gene expression are deemed of interest and may be progressed for further testing.

### Example 2

### Assay for the Modulation of Intracellular Triglycerides

Cryopreserved human primary pre-adipocytes harvested from the subcutaneous adipose tissue of a healthy female are obtained from Zen-Bio (Research Triangle Park, N.C.). Following the manufacturer's instructions, the pre-adipocytes are cultured in Preadipocyte Medium containing DMEM/Ham's F-12 (1:1, v/v), HEPES (pH 7.4), fetal bovine serum, penicillin, streptomycin, and amphotericin B (Zen-Bio), in a humidified 37° C. incubator with 5% CO₂. After reaching 90% confluence, the pre-adipocytes are allowed to differentiate into adipocytes by adding Adipocyte Differentiation Medium containing DMEM/Ham's F-12 (1:1, v/v), HEPES pH 7.4, fetal bovine serum, biotin pantothenate, human insulin, dexamethasone, isobutylmethylxanthine, penicillin, streptomycin, and amphotericin B (Zen-Bio).

To treat adipocytes with potential CPT-1 modulating compounds, a test compound is dissolved in Adipocytes Differentiation Medium and then added into cell culture for 7 days. Untreated adipocytes are used as a control. After 7 days of incubation, Adipocytes Differentiation Medium is replaced with Maintenance Medium containing a test compound, DMEM/Ham's F-12 (1:1, v/v), HEPES pH 7.4, fetal bovine serum, biotin pantothenate, human insulin, dexamethasone, penicillin, streptomycin, and amphotericin B, and the adipocytes continued under incubation for another 7 days. The production of triglycerides in the adipocytes is determined by using a triglyceride assay kit (Zen-Bio). Briefly, adipocytes are rinsed with a wash buffer and lysed in a lysis buffer following medium removal. Intracellular triglycerides are released into the lysis buffer and converted into glycerol-1-phosphate, which is subsequently oxidized to di-hydroxyacetone phosphate and hydrogen peroxide. Hydrogen peroxide is reacted with 4-aminoantipyrine (4-AAP) and sodium N-ethyl-N-(3-sulfopropyl)-m-anisidine (ESPA) to generate a quinoneimine dye, which shows an absorbance maximum at 540 nm. The increase in absorbance at 540 nm is directly proportional to the intracellular levels of triglycerides in the adipocytes. Results are obtained in triplicate and a p-value is determined.

Human adipocytes treated with CPT-modulating compounds will show a significant % difference in intracellular triglyceride levels.

### Example 3

### Procollagen-I ELISA Assay

Human dermal fibroblasts were plated at 5000˜7000 cells/well in 96-well culture plates in supplemented medium (DMEM, 10% Fetal Bovine Serum, 1% Penicillin/Streptomycin and 1% L-Glutamine) for 24 hours in humidified atmosphere of 10% CO₂ at 37° C. The following day, the medium was replaced with fresh medium (DMEM, 10% Fetal Bovine Serum, 1% Penicillin/Streptomycin and 1% L-Glutamine) and Averrhoa carambola (starfruit) leaf extract was added to the wells in triplicate at a concentration of 0.01% and 0.1%. Water was used a vehicle control. Following 72-hour incubation, the plates were removed from the incubator and the conditioned medium from each well was collected for the procollagen-I ELISA.

Collagen production was measured using procollagen type I C-peptide (PIP) EIA kit (Takara Bio, Inc., Japan). Briefly, the conditioned medium was diluted 1:25 in Sample Diluent. 20 l of diluted conditioned medium and 100 microliters of antibody-POD conjugate solution were added to the wells of the Takara ELISA plate. The ELISA plate was incubated at 37° C. for 3 hours before the wells were washed four times with 400 microliters of 1X PBS. At the end of wash, 100 microliters of substrate solution (supplied with kit) was added to the wells and incubated at room temperature for 15 minutes. The reaction was stopped by adding 100 microliters of 1N sulfuric acid to the wells. The absorbance was measured on a spectrophotometer at 450 nm wavelength. The amount of procollagen peptide in the conditioned medium was calculated from the standard curve. The stimulation of collagen production was shown as an increase in collagen over the vehicle control.

Table I below depicts the increased levels of pro-collagen-I by Averrhoa carambola leaf extract in human dermal fibroblasts:

TABLE I
Increased levels of pro-collagen-I by Averrhoa carambola
leaf extract in human dermal fibroblasts

| | | % change over |
|---|---|---|
| | Conc. | vehicle control* |
| Averrhoa carambola | 0.01% | 10.93% |
| (starfruit) leaf extract | 0.1% | 61.86% |

### Example 4

### Consumer Study

A consumer study may be performed to demonstrate that use of a cosmetic cream comprising at least one CPT-1 stimulator reduces the appearance of cellulite after a 4 week treatment regimen of daily treatment (for example, once daily, twice daily, or thrice daily); and/or that at least one CPT-1 inhibitor stimulates lipid production (“lipogenesis”) in the skin and prevents, reduces, ameliorates, and/or eliminates signs of dermatological aging and/or improves the aesthetic appearance of the skin after a 4 week treatment regimen of daily treatment.

### Example 5

### Exemplary Compositions

The cosmetic composition of a cream comprising a CPT-1 modulator for topical application to the skin may be formulated by methods known in the cosmetic arts. The cream comprising at least one CPT-1 modulator may optionally be administered along with a cosmetic composition of a gel for optimal results. Suitable ingredients for such formulations are found in the INCI Ingredient Dictionary and Handbook, 11th Edition (2006), and in the International Cosmetic Ingredient Dictionary and Handbook, 10th Edition (2004), published by the Cosmetic, Toiletry, and Fragrance Association (CTFA), the disclosures of which are hereby incorporated by reference in their entirety.

All references including patent applications and publications cited herein are incorporated herein by reference in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. A method for treating a skin condition characterized by excess lipids, comprising topically applying to skin in need thereof an effective amount of at least one Carnitine Palmitoyl Transferase-1 (CPT-1) stimulator in a cosmetically acceptable vehicle for a time sufficient to improve the appearance of said skin.

2. The method according to claim 1, wherein said at least one CPT-1 stimulator is selected from the group consisting of bezafibrate, fenofibrate, capsaicin, curcumin, docosahexaenoic acid, (−)-epigallocatechin-3-gallate, caffeine, auraptene, R-alpha-lipoic acid, acetyl-L-carnitine, trans-10, cis-12 conjugated linoleic acid, soy isoflavones, L-carnitine, bitter melon, peroxisome proliferator-activated receptor beta/delta agonist GW501516, rexinoids, thiazolidinediones, alpha-linolenic acid, tetrahydro-4-methylene-2R-octyl-5-oxo-3S-furancarboxylic acid (C75), biguanide (buformin), genestein, inhibitors of BHLHB2 proteins, 3,5-dihydroxybenzoic acid derivatives, hydroxamic acid derivatives, and combinations thereof

3. The method according to claim 1, wherein the skin condition is cellulite.

4. The method according to claim 1, wherein the skin condition is acne.

5. The method according to claim 1, wherein the skin condition is oily skin.

6. The method according to claim 1, wherein said at least one CPT-1 stimulator in a cosmetically acceptable vehicle further comprises at least one other anti-lipid agent.

7. The method according to claim 6, wherein said at least one other anti-lipid agent comprises at least one agent selected from the group consisting of a phosphodiesterase inhibitor, an adenylate cyclase activator, a lipolysis stimulator, a beta-adrenergic receptor agonist, an alpha-2-adrenergic receptor antagonist, a xanthine analog, forskolin, a Coleus forskohlii extract, a hawthorne extract, a cola extract, isoproterenol, yohimbine, Ginkgo biloba extract, perilla oil, and combinations thereof

8. The method according to claim 1, wherein said at least one CPT-1 stimulator in a cosmetically acceptable vehicle further comprises at least one collagen and/or elastin stimulator.

9. A method for reducing the re-occurrence of cellulite in an area previously affected by cellulite, comprising topically applying thereto an effective amount of at least one CPT-1 stimulator in a cosmetically acceptable vehicle, for a time sufficient to improve the appearance of said skin.

10. A personal care or cosmetic composition for treating a skin condition characterized by excess lipids, comprising an effective amount of at least one CPT-1 stimulator in a cosmetically acceptable vehicle.

11. The composition according to claim 10, wherein said composition further comprises at least one other anti-lipid agent selected from the group consisting of a xanthine analog, forskolin, a Coleus forskohlii extract, a hawthorne extract, a cola extract, isoproterenol, yohimbine, Ginkgo biloba extract, perilla oil, a phosphodiesterase inhibitor, an adenylate cyclase activator, a lipolysis stimulator, a beta-adrenergic receptor agonist, an alpha-2-adrenergic receptor antagonist, and combinations thereof.

12. The composition according to claim 10, wherein said composition further comprises at least one collagen and/or elastin stimulator.

13. A method for identifying CPT-1 modulators, comprising:
contacting cultured adipocytes with a candidate compound;
measuring CPT-1 expression mRNA from said adipocytes; and
comparing the CPT-1 mRNA expression levels from adipocytes treated with the compound of interest to CPT-1 mRNA levels from untreated control adipoctyes,
wherein a candiate compound which decreases CPT-1 mRNA expression levels is determined to be a CPT-1 inhibitor and a candiate compound which increases CPT-1 mRNA expression levels is determined to be a CPT-1 stimulator.

14. A cosmetic composition comprising a CPT-1 modulator identified by the method of claim 13.

15. The method according to claim 13, wherein said method further comprises:
(i) culturing adipocytes;
(ii) treating one portion of said adipocytes with a compound of interest, and treating another portion of said adipocytes identically but without a compound of interest as an adipocyte negative control, for a time and under conditions sufficient to provide synthesis of triglycerides in said adipocytes;
(iii) lysing said adipocytes and releasing said triglycerides;
(iv) measuring the released triglyceride levels; and
(v) comparing the released triglyceride levels from adipocytes treated with the compound of interest with the released triglyceride levels from control, wherein a suitable active ingredient is identified as a compound of interest which increases or decreases triglyceride levels from lysed adipocytes compared to the control.

16. A cosmetic composition, comprising an active ingredient for the treatment of cellulite identified according to the method of claim 17.

17. A method for treating a skin condition characterized by insufficient subcutaneous lipids, comprising topically applying to skin in need thereof an effective amount of at least one Carnitine Palmitoyl Transferase-1 (CPT-1) inhibitor in a cosmetically acceptable vehicle for a time sufficient to improve the aesthetic appearance of said skin.

18. A method for improving the aesthetic appearance of skin comprising topically applying to skin in need thereof an effective amount of at least one substance capable of modulating Carnitine Palmitoyl Transferase-1 (CPT-1) in human preadipocyte cells in a cosmetically acceptable vehicle for a time sufficient to improve the aesthetic appearance of said skin.

19. A method for imparting an anti-aging benefit to skin comprising topically applying to skin in need thereof an effective amount of at least one substance capable of modulating Carnitine Palmitoyl Transferase-1 (CPT-1) in human preadipocyte cells in a cosmetically acceptable vehicle for a time sufficient to prevent, ameliorate, inhibit and/or reduce signs of dermatological aging of said skin.

20. The method of claim 18, where the improvement in aesthetic appearance is characterized by increased production of procollagen.
